(19) United States
(12) Patent Application Publication
(10) Pub. No.: US 2002/0106716 A1
(43) Pub. Date: Aug. 8, 2002

(54) Device and method for the spectrophotometric analysis of fluids

(76) Inventors: Jean-Pierre Leboeuf, Presles (FR); Francis Therry, Montgeroult (FR)

Correspondence Address: NILLES & NILLES, S.C., FIRSTAR CENTER, SUITE 2000, 777 EAST WISCONSIN AVENUE, MILWAUKEE, WI, 53202-5345, US

(21) Appl. No.: 09/918,311

(22) Filed: Jul. 30, 2001

(30) Foreign Application Priority Data
Feb. 8, 2001 (FR) 01 01740

(51) Int. Cl. C12Q 1/04; G06F 19/00; C12M 1/34

(52) U.S. Cl. 435/34; 702/19; 435/288.5

(57) ABSTRACT

Spectrophotometric analysis device having a feeder circuit of a test stand. The analysis device has two distinct spectrophotometers facing the same test stand, especially in order to be able to cover spectra with wavelengths that are separate and included in the wavelengths on either side of the visible range. A first spectrophotometer covers the spectrum of the near and mean infrared ranges while a second spectrophotometer covers the spectrum of the ultraviolet and visible ranges. This analysis device provides especially for the spectrophotometric determination of the different constituents contained in fluids to be analysed, such as wine, fruit juice or blood.

### BACKGROUND OF THE INVENTION

1. Field of the Invention

An object in the present invention is a device for the spectrophotometric analysis of fluids and a method of analysis implementing said device. It can be used more particularly in the analysis of liquids, especially in order to check the concentration levels of the different constituents of these liquids. The liquids that can be analysed by this type of device are, for example, alcoholic or non-alcoholic beverages or fluids such as blood obtained from the human body or from animals. The term “alcoholic or non-alcoholic beverages” generally refers to grape musts for use in winemaking, musts in fermentation and/or wines. This device and this method can be used to obtain a quantitative determination of the constituents of these liquids. The value of this invention is that it enables a fast and simple quantitative analysis as well as a qualitative analysis of the analysed fluid.

2. Description of the Related Art

In the prior art, the teaching of the document EP-A-0 588 892 is known. This document teaches a method and apparatus for the spectrophotometric determination of aqueous fluids, implementing an interferometer to acquire absorbance spectra of the analysed fluid. A method of this kind determines the concentrations of the constituents of aqueous liquids to be analysed.

The spectrophotometric determination apparatus implements an interferometer to obtain an interferogram, an interferogram being generally called an absorbance spectrum. The absorbance spectrum corresponds to the wavelengths emitted by the apparatus through the liquid and not absorbed in the analysed liquid. Generally, an instrument of this kind emits a spectrum in the infrared wavelengths. To determine constituent elements of a liquid to be tested, an interferogram of the liquid to be tested is compared with interferograms obtained with the same apparatus, using known liquids comprising especially known concentrations of each of the constituents.

To determine the concentration levels of x constituents of a liquid to be tested, at least x wavelengths of an absorbance spectrum obtained with this liquid are taken into account. This gives a system of polynomial equations with at least x equations with x unknowns to be resolved. Indeed, for each wavelength, the absorbance can be expressed as the sum of the absorbance values due, for this wavelength, to each of the constituents. The absorbance values related to each of the constituents are weighted by specific correlation coefficients for each of the wavelengths and each of the constituents.

These correlation coefficients are preferably determined by multiple linear regulations on the basis of absorbance spectra obtained on known reference liquids, whose concentration levels in different constituents are known. The proportions of the different constituents are analysed beforehand by means of other determination methods, for example conventional methods of chemical titration. These correlation coefficients may be also called spectroscopic criteria.

No technique of analysis has been developed in the prior art for the quantitative determination analysis of wines, fermentation musts and grape musts. In particular, no constituent to be analysed in these liquids has been subjected to a determining of correlation coefficients, or of spectroscopic criteria by which it would then be possible, by direct analysis of an interferogram, to determine real concentrations of these constituents in wines or fermentation musts, and/or grape musts.

This prior art raises a problem because preparing the spectroscopic criteria is a lengthy process, and it is not possible to determine them for all the known chemical constituent elements, without making a predetermined choice.

### OBJECTS AND SUMMARY OF THE INVENTION

The usefulness of the invention is that it proposes a device comprising a spectrophotometer emitting in the infrared to automatically and speedily analyse the major chemical constituents to obtain quantitative and qualitative assessment of the wines and of the fermentation musts and/or grape musts. Another useful feature of the invention is that it proposes a device in which these spectroscopic criteria are determined beforehand, for example during the manufacture of the device. Thus, the device is immediately ready for use and immediately, without any other preliminary analyses, gives a result on the concentration levels in different constituents of the analysed liquids.

Indeed, an object of the invention is a device for the analysis of liquid comprising:

a mechanical means to take a sample of a liquid to be analysed and means to convey it before

a spectrophotometric analysis means of the device, this analysis means preferably emitting a light spectrum in the infrared through the sample presented in an analysis cell of this analysis means,

a means for measuring an absorbance spectrum obtained after passage through the sample, this measurement means being linked to

a mathematical processing means, this means comprising a memory in which spectroscopic criteria are recorded, and comprising a computation means to correlate the spectroscopic criteria and the absorbance spectrum so as to determine concentration levels of different constituents,

wherein

the stored spectroscopic criteria enable the automatic determining of the concentration levels of specific constituents of wine and/or grape musts and/or fermenting musts, for example:

gluconic acid concentration revealing the presence of a first microbiological agent and/or

acetaldehyde and/or ethyl acetate concentrations revealing the presence of a second microbiological agent and/or

acetic acid and/or ethyl acetate concentrations revealing the presence of a third microbiological agent and/or

lactic acid concentration revealing the presence of a fourth microbiological agent.

At the same time, an object of the invention is also a method for the spectrophotometric analysis of a liquid comprising the following steps:

a sample of a liquid (2) to be analysed is taken (4, 6), and

it is conveyed (7, 8, 9, 10, 11, 12) into an analysis cell (5) of a means of spectrophotometric analysis (14),

a continuous spectrum is emitted (15) with the analysis means in the infrared through the sample presented,

an absorbance spectrum obtained after passage through the sample is measured (16),

using a mathematical processing means (22), spectroscopic criteria and absorbance spectrum are correlated so as to determine concentration levels of different constituents of this liquid to be analysed,

wherein

in a memory of the mathematical processing means, a recording is made of the spectroscopic criteria by which it is possible to automatically determine at least concentration levels of specific constituents of the wine and/or grape musts and/or fermenting musts, for example:

concentration of gluconic acid revealing the presence of a first microbiological agent, and/or

concentration of acetaldehyde and/or ethyl acetate revealing the presence of a second microbiological agent, and/or

concentration of acetic acid and/or ethyl acetate revealing the presence of a third microbiological agent, and/or

concentration of lactic acid revealing the presence of a fourth microbiological agent.

In other words, the invention pertains to a device for the analysis of liquid comprising:

a mechanical means to take a sample of a liquid to be analysed and means to convey it before

a spectrophotometric analysis means of the device, this analysis means preferably emitting a light spectrum in the infrared through the sample presented in an analysis cell of this analysis means,

a means for measuring an absorbance spectrum obtained after passage through the sample, this measurement means being linked to

a mathematical processing means, this means comprising a memory in which spectroscopic criteria are recorded, and comprising a computation means to correlate the spectroscopic criteria and the absorbance spectrum so as to determine concentration levels of different constituents,

wherein

the stored spectroscopic criteria enable the automatic determining of the concentration levels of specific constituents of wine and/or grape musts and/or fermenting musts, for example:

concentration of a component revealing the presence of de Botrytis cinerea, and/or

concentration of a component revealing the presence of yeasts, and/or

concentration of a component revealing the presence of acetic bacteria and/or

concentration of a component revealing the presence of lactic bacteria.

And the invention also pertains to a device for the spectrophotometric analysis of a fluid comprising a first spectrophotometer, the first spectrophotometer comprising a first light source and a first detector positioned on either side of a first test stand, the first light source emitting in the first range of wavelengths towards the first test stand, the device comprising a second spectrophotometer comprising a second light source and a second detector positioned on either side of a second test stand, the second light source emitting in a second range of wavelengths towards this second test stand.

### BRIEF DESCRIPTION OF THE DRAWING

The invention will be understood more clearly from the following description and the appended figure. This figure is given purely by way of an indication and in a no way restricts the scope of the invention. The figure shows:

FIG. 1: an embodiment of the spectrophotometric and analysis device according to the invention.

### DESCRIPTION OF THE PREFERRED EMBODIMENTS

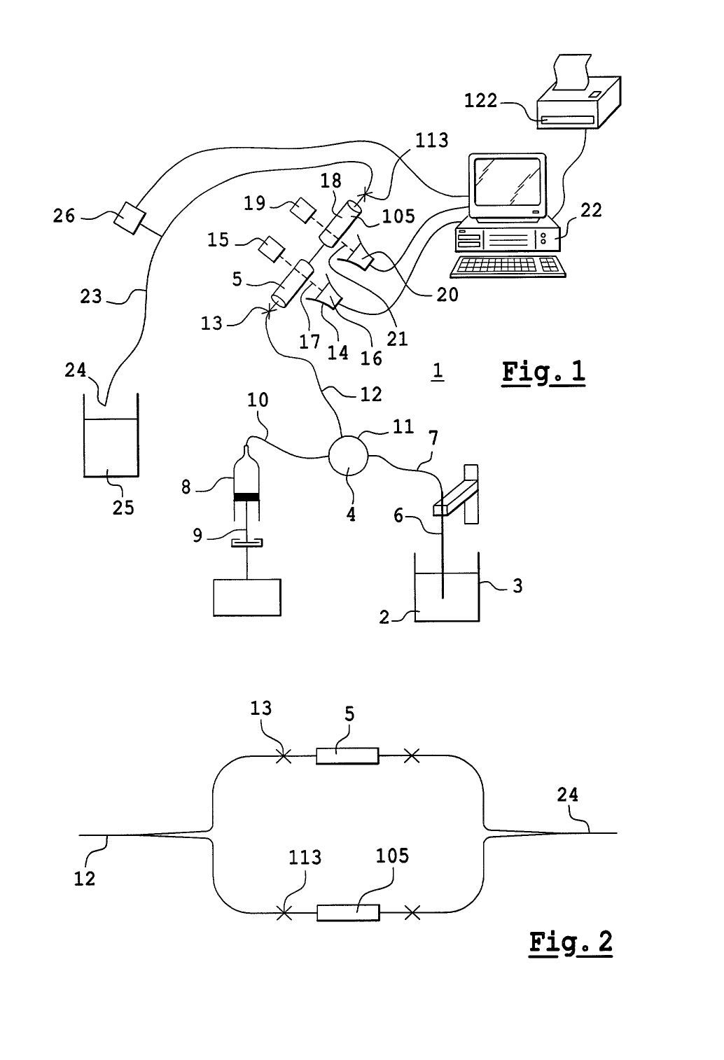

FIG. 1 shows a device 1 according to the invention. The device 1 is used to analyse a fluid 2. The fluid 2 is preferably a liquid. In a preferred example, the device 1 is used for the quantitative determination of the liquids 2 such that the liquids 2 are preferably aqueous alcoholic liquids, for example wines or grape musts and/or fermenting grape musts.

In this case, as shown in the figure, the liquid 2 is contained in a container 3. This container 3 may, for example, contain only a sample of the liquid to be analysed. In this case, the container 3 has a small internal volume. The container 3 may, for example, be positioned on a feeder device that can receive several containers such as 3. The feeder device feeds the device 1 and is, for example, rotational.

The device 1 has a feeder circuit 4 for the test stand 5 of the device 1. The test stand 5 corresponds to a zone at which the fluid 2 is analysed. In one particular embodiment, tire feeder circuit 4 comprises a sample-taking pipette or needle 6 that can be plunged into the container 3. This pipette 6 is connected by means of a first tube 7 to a chamber 8. In one optimised variant, the device 1 has a filtering means between the container 3 and the chamber 8.

In the chamber 8, it is possible to create a momentary depression so as to make the fluid 2 come into this chamber 8 through a pumping means. in a preferred embodiment, the chamber 8 corresponds to an interior cavity of a syringe provided with a piston 9. The shifting of the piston 9 creates momentary depressions in the chamber 8. In one variant, to create a depression by which the fluid 2 can be put into motion, a peristaltic pump is used.

Then, the fluid 2 stored in the chamber 8 is sent by means of a second tube 10 and possibly a three-way valve 11 towards the test stand 5.

In this embodiment, the three-way valve 11 has a first input connected to the first tube 7, a second input connected to the second tube 10 and a third output connected to the third tube 12. This third tube 12 connects the three-way valve 11 precisely to the test stand 5.

Then, through the feeder circuit 4, a continuous and regular flow of the fluid to be analysed is created in the test stand 5. Furthermore, the feeder circuit 4 may also comprise a device to regulate the temperature of the fluid 2 by Peltier effect. In a preferred example, a fraction of the fluid 2 to be analysed in the test stand 5 is momentarily immobilised. To block the fluid 2 in the test stand 5, the tube 12 comprises, for example, two solenoid valves 13 positioned on either side of the test stand 5. Since the flow is even, if the two-solenoid valves 13 are closed simultaneously, a fraction of fluid 2 is blocked in the test stand 5. The internal excess pressure is consequently limited. Indeed, the flow is laminar when the two solenoid valves 13 are closed. Thus, when the liquid 2 is analysed, it shows no microscopic motions at the test stand 5.

To perform these analyses, the device 1 has a first spectrophotometric analysis means or spectrophotometer 14. The first spectrophotometer 14 is positioned so as to face the test stand 5. In particular, it has a first light source 15 emitting towards a first measurement means 16. This measurement means 16 is a detector 16 positioned so as to be facing the emitter 15 in such a way that a light flux 17 emitted by the source 15 crosses an analysis cell of the test stand 5. This first light source 15 emits a continuous spectrum, preferably in a first range of wavelengths. Consequently, the test stand 5 is made with a thickness and out of a material that is specifically suited to the wavelengths emitted by the source 15. Similarly, the technical characteristics of the first detector 16 are adapted.

The first spectrophotometer 14 is a Fourier transform Michelson type interferometer used to obtain continuous absorbance spectra for wavelengths within the infrared range and more particularly in the near infrared and medium infrared ranges, namely in the ranges between 1.5 microns and 2.5 microns and between 2.5 microns and 20 microns respectively.

In this case, the first source 15 is for example a halogen source or a heated filament. And the first detector 16 is then made out of silicon or DTGS.

The medium infrared and near infrared wavelengths can be used especially to determine the proportions of the following components: alcohols, proteins, ethanol, total SO2, CO2, mannitol, arabitol, glycerol, butanediol, sorbitol, methyl-3-Butanol-1, ethyl acetate, acetaldehyde, mesoinositol, α-amino nitrogen, ammonia nitrogen, sugars, reducing sugars, total sugars, glucose, fructose, total acids, volatile acids, organic acids, tartric acids, acetic acid, lactic acid, malic acid, gluconic acid and H30+ions, to assess the pH factor. It can also be used to evaluate the dry extract, density, Brix degree, the mass per unit volume or again the tartric stability of the analysed liquid).

In general, the mean infrared wavelengths make it possible especially to determine the proportions of the organic components. Indeed, for mean infrared and/or near infrared wavelengths, reliable, reproducible and significant results are obtained from known liquid solutions comprising at least one known concentration of one of the constituents referred to here above. Therefore, when a liquid such as 2 is analysed, considering the absorbance values obtained for different specific wavelengths, the concentration levels of these different constituents can be deduced mathematically.

In one variant, the device 1 furthermore has a second spectrophotometer 18. The second spectrophotometer 18 has a second light source 19 and a second detector 20. The second spectrophotometer 18 is then positioned so that it faces a second test stand 105. In this case, the second light source 19 and the second detector 20 are positioned on either side of the test stand 105 so that a light flux 20 emitted by the source 19 crosses the test stand 105. The test stand 105 preferably has two solenoid valves 113 on either side to block the fluid during analysis.

This second spectrophotometer 18 preferably emits light in a second range of wavelengths. To this end, the thickness and the materials constituting the test stand 105 are also specifically matched to the wavelengths emitted by the second source 19. Similarly, the technical characteristics of the second detector 20 are specifically matched to the wavelengths emitted by the second source 19.

This variant has the advantage of neither generating any loss of time nor requiring a double sampling or double handling of the samples. Nor again does it entail any risk of errors between the samples. Furthermore, this method is faster and this twofold spectrophotometric analysis gives greater precision in the determination of the different constituents in the liquid 2.

In a preferred variant, the second spectrophotometer 18 is used to scan a wider spectrum of wavelengths so as to obtain an absorbance spectrum for wavelengths included in a range preferably distinct from the first range of wavelengths. The second spectrophotometer 18 gives absorbance spectra for wavelengths within the ultraviolet and visible zones. It is used to scan a spectrum of wavelengths within a range from 0.1 microns to 1 micron.

The second source 19 is for example a deuterium lamp or tungsten lamp. In this case, the second detector 20 is an array of diodes or an array of CCD detectors.

The ultraviolet and visible wavelengths can be used especially to determine the proportions of the following constituents: H30+ions, acids, total acids, the volatile acids, acetic acid, tartric acid, gluconic acid, sorbic acid, polyphenols, tannins, proteins, free SO2, total SO2, anthocyanes, nitrates, dissolved oxygen and volatile constituents. Thus, greater precision is obtained in the quantitative determination of the different acid constituents, total polyphenols, anthocyanes, tartric acid, free SO2, total SO2 and sorbic acid present in the fluid 2 to be analysed.

The width of the wavelength emission spectrum covered by the first spectrophotometer 14 and the second spectrophotometer 18 is jointly 0.1 microns to 25 microns.

On the whole, these quantitative analyses and measurements are useful for assessing the qualitative characteristics of the tested liquids 2 such as wine or grape musts or, again, fermenting musts. For example, for a wine, these quantitative analyses must be made at different stages of its preparation. For example, the ripeness of the grapes is checked before the grapes are gathered. An assessment is made of the level of contamination of the analysed liquid 2 by rot type bacteria. T his assessment is made during the grape-picking process or during the fermentation of the grape juice. Finally, the fermenting musts and the wine at the end of the fermentation when it is put on the market are regularly checked. They can be used to make comprehensive checks of ripeness and follow up the potential of the final product.

In the invention, it is planned especially to use absorbance spectra given by the first spectrophotometer 14 and possibly given by the second spectrophotometer 18 to assess the concentration levels of different constituents likely to reveal the presence of certain bacteria or yeasts.

For example, from the concentration in gluconic acid, it is sought to reveal the presence of a first microbiological agent in the liquid 2. Since this first microbiological agent is Botrytis cinerea , it may also be revealed by the concentration levels of mannitol or in sorbital, present in this liquid 2.

From the levels of concentration in acetaldehyde and/or ethyl acetate, it is sought to reveal the presence of a second microbiological agent in the liquid 2. Since this second microbiological agent consists of yeasts, it may also be revealed by concentration levels of arabitol, 2,3-butanediol, methyl-3-butanol-1, glycerol and/or isoamyl acetate present in this liquid 2.

From the levels of concentration in acetic acid and/or ethyl acetate, it is sought to reveal the presence of a third microbiological agent in the liquid 2. Since this second microbiological agent consists of yeasts, it may also be revealed by concentration levels of arabitol, 2,3-butanediol, methyl-3-butanol-1, glycerol and/or isoamyl acetate present in this liquid 2.

From the level of concentration in lactic acid, it is sought to reveal the presence of a fourth microbiological agent in the liquid 2. Since this fourth microbiological agent consists of lactic bacteria, it may also be revealed by concentration levels of mannitol and/or 2,3-butanediol present in this liquid 2.

Among these bacteria, it is sought especially to reveal at least one of the following microbiological species in the analysed liquid 2: Botrytis cinerea, lactic bacteria and/or acetic bacteria.

For example, to reveal the presence of Botrytis cinerea, the gluconic acid concentration in the liquid 2 is considered. In one improvement, it is also possible to reveal this presence of Botrytis cinerea by concentration levels of mannitol or sorbital.

Similarly, to reveal the presence of yeast, acetic acid and/or ethyl acetate concentration levels in the liquid 2 are considered. In one improvement, this presence of yeasts can also be revealed by considering the concentration levels of arabitol, 2,3-butanediol, methyl-3-butanol-1, glycerol and/or isoamyl acetate present in this liquid 2.

To reveal the presence of acetic bacteria, the acetic acid and/or ethyl acetate concentration levels in the liquid 2 are considered. In one improvement, this presence of acetic bacteria can also be revealed by considering the concentration of 2,3-butanediol.

Finally, to reveal the presence of lactic bacteria, the concentration of lactic acid in the liquid 2 is considered. In one improvement, this presence of lactic bacteria can also be revealed by considering the concentration levels of mannitol and/or 2,3-butanediol.

Furthermore the characteristics sought or permitted by this type of spectrophotometer, for the precise quantitative evaluation of liquids such as wine, grape musts or fermenting musts, include the alcohol percentage which is a function of the alcohol concentration, the total acidity, the volatile acidity, the coloring intensity, the Folin index, the assimilable nitrogen content and the concentration levels of citric acid, ascorbic acid, acetaldehyde, saccharose and ammonia.

The utility of carrying out a quantitative analysis of the difference sugars of the liquid lies in the fact that it can be used to ascertain that the macerating juice has not been illicitly enriched by means of a sugar external to the initial grapes.

Furthermore the usefulness of obtaining spectra in the ultraviolet and visible regions lies in the fact that it enables the performance of official reference methods to measure the optical density (DO280), and hence to measure the color, for example on the basis of two or three wavelengths (0.420 micron, 0.520 micron, 0.620 micron).

In a preferred embodiment, the absorbance spectra measured by the detectors 16 and 20 respectively are preferably processed by a mathematical processing means 22, preferably placed in a computer. This mathematical processing means 22 comprises a computation means to correlate the absorbance values read by the detector 16 and, possibly, those read by the detector 20, with reference absorbance spectra. It searches for the best spectral coincidences to make a precise determination of the proportions of the different constituent elements.

The reference absorbance spectra have been obtained during a preliminary calibration step and are stored in a memory of the mathematical processing means 22. Similarly, the correlation coefficients of each specific pair (wavelength, component) are also stored in this memory. They may be stored, for example, in the form of a matrix of calibration data. Indeed, the computer 22 comprises calibration data for each of the components that can be analysed from an interferogram, so that they can be subjected to quantitative analysis in samples of the liquid 2 to be tested.

The computer 22 collects all the spectra obtained by the spectrophotometer 14 and/or the spectrophotometer 18. From all the results of absorbance provided and, especially, for specific wavelengths, the determination of the constituents is made. The mathematical methods, for example methods of the PLS (Partial Least Squares) or MLR type are applied, preferably simultaneously, to this set of data given by the spectra obtained in the near and medium infrared, and/or the spectra obtained in the ultraviolet and the visible ranges.

The computer 22 preferably has a screen to make results given by this computer 22 available to a user placed before this screen. For example, this computer 22 may be connected to a printer 122 to print out the results given by the computer 22 in a chosen format.

Finally, once the wavelength spectra that can be emitted by each of the two light sources 15 and 19 have been scanned, or even before the mathematical processing is completed, it is planned to open the solenoid valves 13 and 113 so as to release the fluid 2, which has now been analysed. To make sure that the fluid 2 is let out, it may be planned to push the piston 9 even further so as to definitively push all the liquid into an outlet tube 23. However, in one variant, a suction pump can also be provided at one end 24 of this outlet tube 23. In a preferred embodiment, the outlet tube 23 opens into a waste receptacle 25. The waste receptacle 25 receives the different samples coming from fluids to be analysed such as 2.

If the spectrophotometers are arranged in series (FIG. 1), there will be only one outlet tube 23. However, if they are arranged in parallel (FIG. 2), either each test stand is connected to its own outlet tube, each tube leading to a waste receptacle, or the two tubes meet to form one and the same extremity. 24.

In a particular embodiment, it is planned that the analysis device 1 will also include a probe 26 to measure the conductivity of the fluid 2 to be analysed. This probe 26 is preferably also connected to the computer 22. The data obtained by the probe 26 makes it possible to immediately deduce the oxidation-reduction potential of the fluid 2. This probe 26 is used solely for the analysis of liquid type fluids 2.

In another mode of exploitation, the results given by the interferometers 14 and possibly another interferometer 18 or probe 26 can be used to create a quality index. These results pertain to the concentration levels of different constituents, especially the concentration levels of constituents revealing the presence of Botrytis cinerea, lactic bacteria, acetic bacteria and/or yeasts, the pH value, the color, the oxidation-reduction potential, the coloring intensity, the Folin index, and/or the density of the analysed liquid 2, all these results forming a group of characteristic parameters.

This quality index is defined from the mathematical processing means 22, used firstly to select the parameters that have to be included in this quality index. Secondly, the mathematical processing means 22 enable the assigning of scale of points to each of the parameters selected, leaving the user free to set the values of these points, in doing so independently for each parameter.

Finally, after having allowed for a specific definition of this quality index, the computation means automatically apply the scale of points to each of the selected parameters and then use a planned computation rule of the quality index. For example, this rule is the addition of the points obtained by each of the parameters selected.

Then, the interpretative exploitation of the quality index enables the speedy and easy comparison of the analysed liquids such as the liquid 2 with one another. For example it is possible to establish a quality index representing the undesired level of rot for the wine, in taking account especially of the concentration levels of constituents revealing the presence of this rot, namely the presence of Botrytis cinerea, lactic bacteria, acetic bacteria and/or yeasts. This quality index signifying the rot rate may also be set by assigning a highly discriminating scale of points to these constituents.

Furthermore, it is possible to create any type of quality index. For example, in another index, the color and the concentration levels of polyphenols and anthocyanes are considered so as to determine an index of commercial quality of the product and assess its potential. These parameters are tracked mainly as and when the winemaking method takes place. It makes it possible for example to know which techniques were used during the crushing of the grapes and to follow the progress during the maceration.

Should the fluid 2 to be analysed be human or animal blood, the two spectrophotometers 14 and 18 are used for spectrophotometric determination of the following constituents: glucose, cholesterol, creatine, phosphatases, GOT and GPT transaminases, urea, uric acid, phospholipids, total proteins, HDL, LDL, total lipids, triglycerides and gamma GT.

What is claimed is:

1. A device for the analysis of liquid comprising:
a mechanical means to take a sample of a liquid to be analysed and means to convey it before
a spectrophotometric analysis means of the device, this analysis means preferably emitting a light spectrum in the infrared through the sample presented in an analysis cell of this analysis means,
a means for measuring an absorbance spectrum obtained after passage through the sample, this measurement means being linked to
a mathematical processing means, this means comprising a memory in which spectroscopic criteria are recorded, and comprising a computation means to correlate the spectroscopic criteria and the absorbance spectrum so as to determine concentration levels of different constituents,
wherein
the stored spectroscopic criteria enable the automatic determining of the concentration levels of specific constituents of wine and/or grape musts and/or fermenting musts, for example:
gluconic acid concentration revealing the presence of a first microbiological agent and/or
acetaldehyde or ethyl acetate concentration revealing the presence of a second microbiological agent and/or
acetic acid or ethyl acetate concentration revealing the presence of a third microbiological agent and/or
lactic acid concentration revealing the presence of a fourth microbiological agent.

2. A device according to claim 1, wherein the first microbiological agent is Botrytis cinerea, and can also be revealed as the case may be by concentration levels of mannitol, or sorbitol present in the liquid.

3. A device according to claim 1, wherein the second microbiological agent consists of yeasts, and can also be revealed, as the case may be, by concentration levels of arabitol, 2,3-butanediol, methyl-3-butanol-1, -glycerol and/or isoamyl acetate present in the liquid.

4. A device according to claim 1, wherein the third microbiological agent consists of acetic bacteria and may also be revealed, as the case may be, by a concentration of 2,3-butanediol.

5. A device according to claim 1, wherein the fourth microbiological agent consists of lactic bacteria, and may also be revealed, as the case may be, by concentration levels of mannitol and/or 2,3-butanediol.

6. A device according to claim 1, comprising a second means of spectrophotometric analysis, this second means of analysis preferably emitting a light spectrum in the visible and the ultraviolet domains through the sample presented in the second test stand, and comprising a second means for the measurement of an absorbance spectrum obtained after passage through the sample, this measurement means being connected to the mathematical processing means.

7. A device according to claim 1, comprising means to create a quality index from the results of the mathematical processing means.

8. A device according to claim 7, wherein the means for creating the quality index comprise
a means to select the concentration levels of the components to be considered in this index, and
a means to assign each of these concentration levels a scale of points as a function of the value of the concentration.

9. A method for the spectrophotometric analysis of a liquid comprising the following steps:
a sample of a liquid to be analysed is taken, and
it is conveyed into an analysis cell of a means of spectrophotometric analysis,
a continuous spectrum is emitted with the analysis means in the infrared through the sample presented,
an absorbance spectrum obtained after passage through the sample is measured,
using a mathematical processing means, spectroscopic criteria and absorbance spectrum are correlated so as to determine concentration levels of different constituents of this liquid to be analysed,
wherein
in a memory of the mathematical processing means, a recording is made of the spectroscopic criteria by which it is possible to automatically determine at least concentration levels of specific constituents of the wine and/or grape musts and/or fermenting musts, for example:
concentration of gluconic acid revealing the presence of a first microbiological agent, and/or
concentration of acetaldehyde and/or ethyl acetate revealing the presence of a second microbiological agent, and/or
concentration of acetic acid and/or ethyl acetate revealing the presence of a third microbiological agent, and/or
concentration of lactic acid revealing the presence of a fourth microbiological agent.

10. A method according to claim 9 wherein
the analysed liquid is discharged into a waste receptacle.

11. A method according to claim 9 wherein
the automatically determined concentration levels of the components are displayed on a computer screen or printed.

12. A method according to claim 9 wherein
a quality index is created from the results of the mathematical processing means,
to this end, the concentration levels of the constituents to be considered in this index are selected and each of these concentration levels is assigned a scale of points as a function of the value of the concentration.

13. A method according to claim 9 wherein
the sample is taken into a second analysis cell of a second means of spectrophotometric analysis,
a continuous spectrum is emitted with the analysis means in the infrared and the visible domains through the sample presented,
an absorbance spectrum obtained after passage through the sample is measured,
using the mathematical processing means, spectroscopic criteria and absorbance spectrum are correlated so as to determine concentration levels of different constituents of this liquid to be analysed,

14. A method according to claim 9 wherein the first microbiological agent is Botrytis cinerea and wherein
a recording is made, in a memory of the mathematical processing means, of the spectroscopic criteria enabling the automatic determining of the concentration levels of mannitol and/or sorbitol present in the liquid in order to reveal it.

15. A method according to claim 9, wherein the second microbiological agent consists of yeasts, and wherein
a recording is made, in the memory of the mathematical processing means, of the spectroscopic criteria enabling the automatic determining of the concentration levels in arabitol, 2,3-butanediol, methyl-3-butanol-1, glycerol and/or isoamyl acetate present in the liquid in order to reveal them.

16. A method according to claim 9, wherein the third microbiological agent consists of acetic bacteria, and wherein
a recording is made, in the memory of the mathematical processing means, of the spectroscopic criteria enabling the automatic determining of the concentration of 2,3-butanediol present in the liquid in order to reveal it.

17. A device for the analysis of liquid comprising:
a mechanical means to take a sample of a liquid to be analysed and means to convey it before
a spectrophotometric analysis means of the device, this analysis means preferably emitting a light spectrum in the infrared through the sample presented in an analysis cell of this analysis means,
a means for measuring an absorbance spectrum obtained after passage through the sample, this measurement means being linked to
a mathematical processing means, this means comprising a memory in which spectroscopic criteria are recorded, and comprising a computation means to correlate the spectroscopic criteria and the absorbance spectrum so as to determine concentration levels of different constituents,
wherein
the stored spectroscopic criteria enable the automatic determining of the concentration levels of specific constituents of wine and/or grape musts and/or fermenting musts, for example:
concentration of a component revealing the presence of de Botrytis cinerea, and/or
concentration of a component revealing the presence of yeasts, and/or
concentration of a component revealing the presence of acetic bacteria and/or
concentration of a component revealing the presence of lactic bacteria.

18. A device according to claim 17 comprising a second means of spectrophotometric analysis, this second means of analysis preferably emitting a light spectrum in the visible and the ultraviolet domains through the sample presented in second test stand, and comprising a second means for the measurement of an absorbance spectrum obtained after passage through the sample, this measurement means being connected to the mathematical processing means.

19. A device according to claim 17, comprising means to create a quality index from the results of the mathematical processing means.

20. A device according to claim 19, wherein the means for creating the index of quality comprise:
a means to select the concentration levels of the components to be considered in this index, and
a means to assign each of these concentration levels a scale of points as a function of the value of the concentration.

21. A device according to claim 17 wherein, to reveal the presence of Botrytis cinerea, the concentration of gluconic acid present in the liquid and, if necessary, the concentration levels of mannitol, and/or of sorbitol present are considered.

22. A device according to claim 17 wherein, to reveal the presence of yeasts, the concentration levels of acetaldehyde and/or ethyl acetate present in the liquid and, as the case may be, the concentration levels of arabitol, 2,3-butanediol, methyl-3-butanol-1, glycerol and/or isoamyl acetate present are considered.

23. A device according to claim 17 wherein, to reveal the presence of acetic bacteria, the concentration levels of acetic acid and/or ethyl acetate present in the liquid and, as the case may be, the concentration of 2,3-butanediol are considered.

24. A device according to claim 17 wherein, to reveal the presence of lactic bacteria, the concentration level of lactic acid present in the liquid and, as the case may be, the concentration levels of mannitol and/or 2,3-butanediol are considered.

25. A device for the spectrophotometric analysis of a fluid (2) comprising a first spectrophotometer, the first spectrophotometer comprising a first light source and a first detector positioned on either side of a first test stand, the first light source emitting in the first range of wavelengths towards the first test stand, the device comprising a second spectrophotometer [(18)] comprising a second light source and a second detector positioned on either side of a second test stand, the second light source emitting in a second range of wavelengths towards this second test stand.

26. A device according to claim 24 wherein the second range of wavelengths corresponds to an ultra-violet and/or visible range of wavelengths.

27. A device according to claim 24, wherein the first light source and the second light source may have a joint emission spectrum with wavelengths ranging from 0.1 micrometers and 20 micrometers.

28. A device according to claim 24, comprising a probe to measure the conductivity of the fluid.

29. A device according to claim 24, comprising a mathematical processing means to process absorbance values given by the two spectrophotometers.

30. A device according to claim 24, wherein the two spectrophotometers are placed in series.

31. A device according to claim 24, wherein the two spectrophotometers are placed in parallel.

32. A device according to claim 24, wherein the liquid to be analysed is an alcoholic or non-alcoholic aqueous liquid, or a human or animal liquid.

33. A method according to claim 9, wherein the fourth microbiological agent consists of lactic bacteria, and wherein
a recording is made, in the memory of the mathematical processing means, of the spectroscopic criteria enabling the automatic determining of the concentration levels of mannitol and/or 2,3-butanediol present in the liquid in order to reveal them.

34. A device according to claim 24 wherein the first range of wavelengths corresponds to a mean infrared and/or near infrared range of wavelengths.
